(19) United States
(12) Patent Application Publication
(10) Pub. No.: US 2023/0092973 A1
(43) Pub. Date: Mar. 23, 2023

(54) COMPUTATIONAL CLASSIFICATION BASED ON DNA SEQUENCE SIGNATURES

(71) Applicant: International Business Machines Corporation, Armonk, NY (US)

(72) Inventors: RITESH VIJAY KRISHNA, Sale (GB); LAURA-JAYNE GARDINER, Wirral (GB)

(21) Appl. No.: 17/482,575

(22) Filed: Sep. 23, 2021

(51) Int. Cl. G16B 40/20; G06N 3/04; G16B 20/20

(52) U.S. Cl. CPC G16B 40/20; G06N 3/04; G16B 20/20

(57) ABSTRACT

A processor may receive DNA data associated with a DNA sequence. The processor may classify the DNA sequence as exhibiting circadian behavior utilizing a graph deep learning algorithm. The graph deep learning algorithm may be trained utilizing a combination of features of a human interactome, features of DNA sequences associated with genes identified as exhibiting circadian behavior, and features of DNA sequences associated with genes identified as not exhibiting circadian behavior.

### BACKGROUND

The present disclosure relates generally to the field of gene classification, and more specifically to classification of genes based on DNA sequence signatures utilizing graph machine learning algorithms.

The circadian clock is an internal molecular 24-hour timer that is a critical adaptation to life on Earth. It temporally orchestrates physiology, biochemistry and metabolism across the day/night cycle e.g., core body temperature, brain wave activity, cardiovascular/respiratory function, coagulation and immunity. Disruption of the clock may impact sleep, attention span, and mental health, with long term health consequences ranging from metabolic dysfunction (that can impact treatment response) to cancer.

The circadian clock is a transcriptional regulatory network which drives complex and robust patterns of temporal gene expression. Genes that are part of this network are defined as having a specific profile of gene expression. Circadian gene expression rhythms reflect a variety of waveform shapes with a characteristic periodicity of about 24 hours. Methods exist for identifying these rhythms from transcriptomic time course datasets. However, understanding such complex transcriptional regulatory systems is limited by the ability to assay them, requiring the generation of long, high-resolution, time-series transcriptomic datasets.

### SUMMARY

Embodiments of the present disclosure include a method, computer program product, and system for coordinating source code commits. A processor may receive DNA data associated with a DNA sequence. The processor may classify the DNA sequence as exhibiting circadian behavior utilizing a graph deep learning algorithm. The graph deep learning algorithm may be trained utilizing a combination of features of a human interactome, features of DNA sequences associated with genes identified as exhibiting circadian behavior, and features of DNA sequences associated with genes identified as not exhibiting circadian behavior.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 is a block diagram of an exemplary system for classification of genes based on DNA sequence signatures, in accordance with aspects of the present disclosure.

FIG. 2 is a flowchart of an exemplary method system for classification of genes based on DNA sequence signatures, in accordance with aspects of the present disclosure.

FIG. 3A illustrates a cloud computing environment, in accordance with aspects of the present disclosure.

FIG. 3B illustrates abstraction model layers, in accordance with aspects of the present disclosure.

FIG. 4 illustrates a high-level block diagram of an example computer system that may be used in implementing one or more of the methods, tools, and modules, and any related functions, described herein, in accordance with aspects of the present disclosure.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

Aspects of the present disclosure relate generally to the field of gene classification, and more specifically to classification of genes utilizing graph machine learning algorithms based on DNA sequence signatures associated with circadian rhythms. While the present disclosure is not necessarily limited to such applications, various aspects of the disclosure may be appreciated through a discussion of various examples using this context.

In some embodiments, a processor may receive DNA data associated with a DNA sequence. In some embodiments, the DNA data may relate to a sequence of human DNA. In some embodiments, the DNA sequence may include a sequence of DNA associated with a gene. In some embodiments, the gene may be a naturally occurring gene. In some embodiments, the gene may include one or more single nucleotide polymorphisms (“SNPs”). In some embodiments, the DNA data may include a k-mer (e.g., DNA ‘words’ of length k) spectrum of the DNA sequence. As an example, for SNP altered genes, DNA sequences may be generated that contain the mutation at the appropriate loci. The modified DNA sequence may be converted into a k-mer spectrum and generate binary features that may be used for classification.

In some embodiments, the processor may classify the DNA sequence as exhibiting circadian behavior utilizing a graph deep learning algorithm. In some embodiments, the graph deep learning algorithm may be trained to classify DNA sequences as exhibiting circadian behavior utilizing a combination of features of a human interactome, features of DNA sequences associated with genes identified as exhibiting circadian behavior, and features of DNA sequences associated with genes identified as not exhibiting circadian behavior. In some embodiments, features of animal or plant interactomes may be additional used or used in substitution of the human interactome.

In some embodiments, the graph deep learning algorithm may include frameworks for both node and graph focused tasks, including techniques such as graph convolutional networks, graph attention networks, etc.

In some embodiments, the DNA sequences of genes identified as exhibiting circadian behavior and the DNA sequences of genes identified as not exhibiting circadian behavior may be identified from data obtained from the results of temporal transcriptomic experiments. As an example, databases such as Gene ATLAS, UNIPROT, CircaDB may be used to create a list of known circadian genes across a list of tissues. The databases may also be used to filter for a list of genes that are known to be non-circadian (e.g., never observed as circadian in any tissues). In some embodiments, the size of the set of genes identified as circadian and the size of the set of genes identified as non-circadian may be adjusted so that there are equal numbers of genes in each set. In some embodiments, the DNA sequences of the genes in both sets may be obtained.

As an example, genes exhibiting circadian behavior that appeared in one or more organ with a P-value<a threshold (e.g., 0.05), False Discovery Rate (“FDR”)<a threshold (e.g., 0.05), minimum relative amplitude (rAMP)>a threshold (e.g., 0.1), and an R2>a threshold (e.g., 0.1) may be identified as genes exhibiting circadian behavior. DNA sequences associated with genes not exhibiting circadian behavior may be identified by obtaining a full gene list (e.g., from the GRCh38 human reference genome) and removing all genes that may be identified as circadian genes using relaxed thresholds (e.g., P-value<0.95, FDR<0.95, rAMP>0.1 and an R2>0.1). In some embodiments, a k-mer spectrum may be created for the DNA sequences of each gene that is identified as exhibiting circadian behavior. In some embodiments, a k-mer spectrum may be created for the DNA sequences of each gene that is identified as not exhibiting circadian behavior. As an example, for each gene in both the set of genes identified as circadian and the set of genes identified as non-circadian, a binary matrix (e.g., elements 0 and 1) may be created to create a [1×n] feature, where n is the total number of k-mers in both sets. In some embodiments, the k-mer profiles may be generated de-novo for the mRNA and promoter sequences associated with each gene. As an example, every possible k-mer of length 6 bp (base pair) may be counted in each transcript and promoter.

In some embodiments, the graph deep learning algorithm (e.g., a graph convolutional network) may be trained utilizing a human interactome. In some embodiments, elements of DNA sequences identified as exhibiting circadian behavior and elements of DNA sequences identified as not exhibiting circadian behavior may be mapped to the human interactome. For example, the elements of the set containing all circadian genes and the set containing all non-circadian genes may be mapped on the human protein interactome obtained from a database. The non-mapping elements and their connections may be dropped from the human protein interactome. In some embodiments, the human protein interactome, as modified, may be converted into a connected graph and translated to an adjacency matrix representation.

In some embodiments, while training the machine learning model for gene classification, the adjacency matrix and the feature representations of the gene feature matrix may be used to combine the gene features with the topology of the underlying interactome (e.g., by treating them as node features and graph structure). For example, the convolution operation of the graphical convolutional network may be specified as H=f(LXW), where L=\hat{D}^{-1/2}Â\hat{D}^{-1/2} with Â=A+I with I being identity matrix, \hat{D} being the degree matrix of Â, W as the trainable weight matrix, H as the updated feature matrix and f being a non-linear activation function, and A being the interactome converted into a connected graph and translated in an adjacency matrix representation.

In some embodiments, training the graph deep learning algorithm may include: identifying circadian genes (e.g., genes exhibiting circadian behavior), obtaining DNA sequences associated with the circadian genes, and translating the DNA sequences associated with the circadian genes to a representation suitable for machine learning. In some embodiments, training the graphical convolutional network may further include: identifying non-circadian genes, obtaining DNA sequences associated with the non-circadian genes (e.g., genes not exhibiting circadian behavior), and translating the DNA sequences associated with the circadian genes to a representation suitable for machine learning. In some embodiments, training the graphical convolutional network may further include: generating a mapping of the DNA sequences associated with circadian genes and DNA sequences associated with non-circadian genes to the human interactome. In some embodiments, training the graphical convolutional network may further include: translating the generated mapping to a representation suitable for machine learning.

In some embodiments, the disclosed processes may be utilized to conduct mass screenings of simulated SNP altered profiles. For example, a large combination of SNP alterations may be generated for each sequence for genes of interest to simulate a population of altered gene profiles. The disclosed process may be used to categorize each profile as circadian or not circadian.

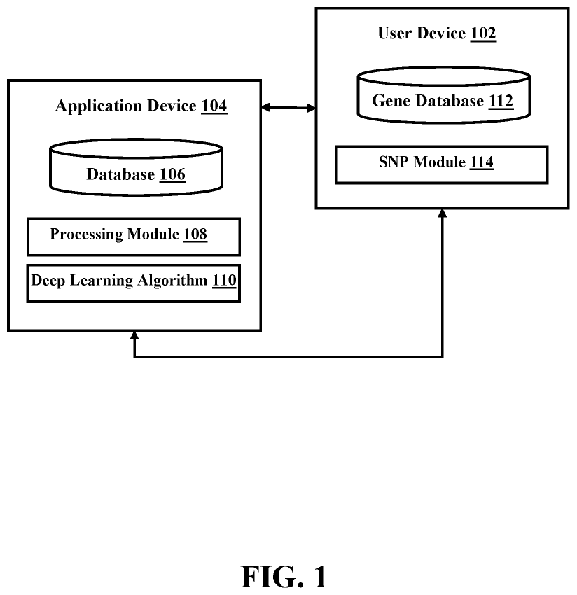

Referring now to FIG. 1, a block diagram of a system 100 for classification of genes based on DNA sequence signatures is illustrated. System 100 includes a user device 102 and an application device 104. The user device 102 is configured to be in communication with the application device 104. The application device 104 includes a database 106, a processing module 108, and a deep learning algorithm 110. In some embodiments, the user device 102 and the application device 104 may be any devices that contain a processor configured to perform one or more of the functions or steps described in this disclosure.

In some embodiments, the deep learning algorithm 110 is a graph deep learning algorithm that classifies genes as exhibiting circadian behavior or not exhibiting circadian behavior. DNA data that is associated with a DNA sequence may be received from the gene database 112 (e.g., a query gene database including SNP altered DNA sequences) of the user device 102. In some embodiments, the DNA sequence may have had a SNP introduced into it using the SNP module 114.

In some embodiments, the deep learning algorithm 110 of the system device 104 may have been trained using k-mer spectra representing the DNA sequences associated with genes identified as exhibiting circadian behavior and k-mer spectra representing the DNA sequences associated with genes identified as not exhibiting circadian behavior. The k-mer spectra are generated using processing module 108. The DNA sequences associated with genes identified as exhibiting circadian behavior and the DNA sequences associated with genes identified as not exhibiting circadian behavior may be obtained from database 106 of system device 104. In some embodiments, the deep learning algorithm 110 of the system device 104 may have been trained using a human interactome having elements of DNA sequences associated with genes identified as exhibiting circadian behavior and elements of DNA sequences associated with genes identified as not exhibiting circadian behavior mapped to the human interactome. The human interactome is obtained from database 106, and the elements of DNA sequences associated with genes identified as exhibiting circadian behavior and elements of DNA sequences associated with genes identified as not exhibiting circadian behavior are mapped to the human interactome by the processing module 108.

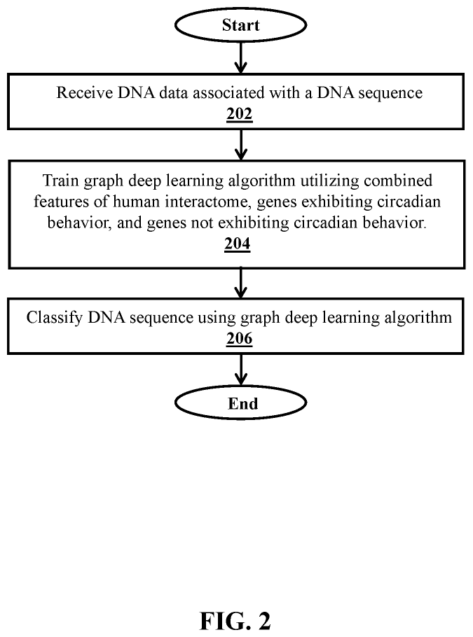

Referring now to FIG. 2, illustrated is a flowchart of an exemplary method 200 for classification of genes based on DNA sequence signatures, in accordance with embodiments of the present disclosure. In some embodiments, a processor of a system may perform the operations of the method 200. In some embodiments, method 200 begins at operation 202. At operation 202, the processor receives DNA data associated with a DNA sequence. In some embodiments, method 200 proceeds to operation 204, where the processor trains a graph deep learning algorithm utilizing a combination of features of a human interactome, features of DNA sequences associated with genes identified as exhibiting circadian behavior, and features of DNA sequences associated with genes identified as not exhibiting circadian behavior. In some embodiments, method 200 proceeds to operation 206, where the processor classifies the DNA sequence as exhibiting circadian behavior utilizing a graph deep learning algorithm.

As discussed in more detail herein, it is contemplated that some or all of the operations of the method 200 may be performed in alternative orders or may not be performed at all; furthermore, multiple operations may occur at the same time or as an internal part of a larger process.

It is to be understood that although this disclosure includes a detailed description on cloud computing, implementation of the teachings recited herein are not limited to a cloud computing environment. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of portion independence in that the consumer generally has no control or knowledge over the exact portion of the provided resources but may be able to specify portion at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

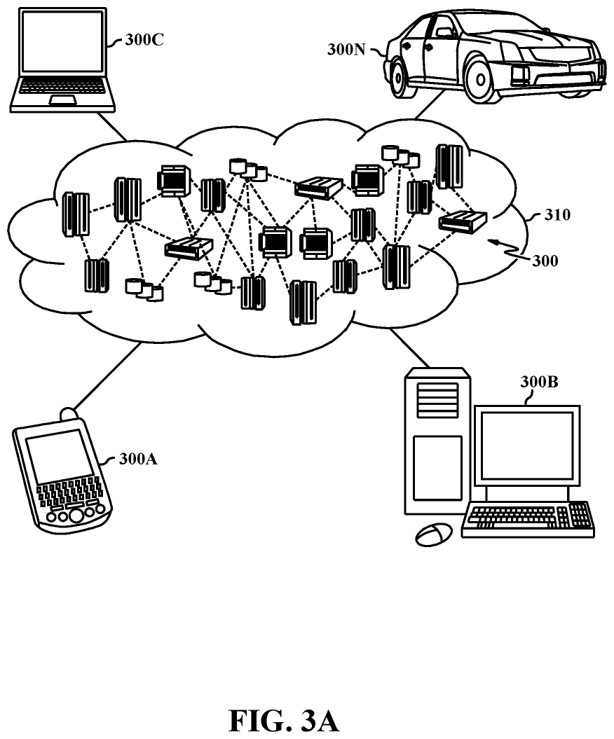

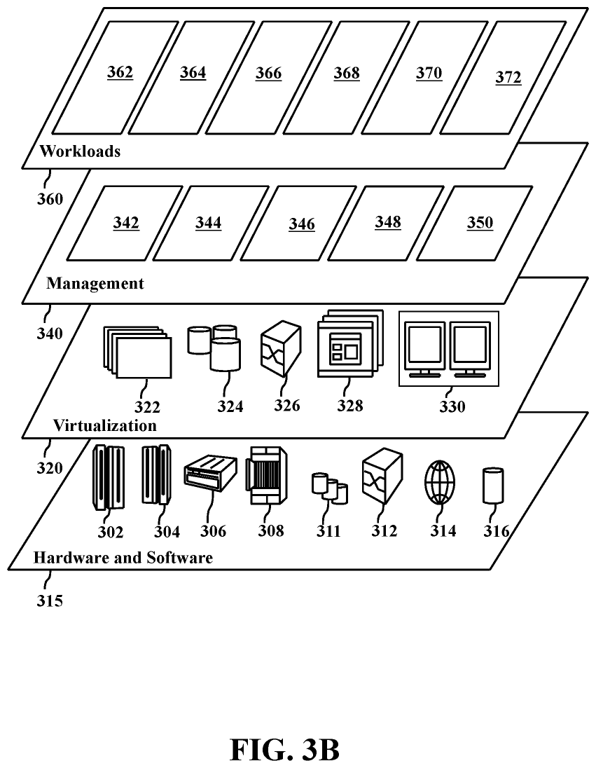

FIG. 3A, illustrated is a cloud computing environment 310 is depicted. As shown, cloud computing environment 310 includes one or more cloud computing nodes 300 with which local computing devices used by cloud consumers, such as, for example, personal digital assistant (PDA) or cellular telephone 300A, desktop computer 300B, laptop computer 300C, and/or automobile computer system 300N may communicate. Nodes 300 may communicate with one another. They may be grouped (not shown) physically or virtually, in one or more networks, such as Private, Community, Public, or Hybrid clouds as described hereinabove, or a combination thereof.

This allows cloud computing environment 310 to offer infrastructure, platforms and/or software as services for which a cloud consumer does not need to maintain resources on a local computing device. It is understood that the types of computing devices 300A-N shown in FIG. 3A are intended to be illustrative only and that computing nodes 300 and cloud computing environment 310 can communicate with any type of computerized device over any type of network and/or network addressable connection (e.g., using a web browser).

FIG. 3B, illustrated is a set of functional abstraction layers provided by cloud computing environment 310 (FIG. 3A) is shown. It should be understood in advance that the components, layers, and functions shown in FIG. 3B are intended to be illustrative only and embodiments of the disclosure are not limited thereto. As depicted below, the following layers and corresponding functions are provided.

Hardware and software layer 315 includes hardware and software components. Examples of hardware components include: mainframes 302; RISC (Reduced Instruction Set Computer) architecture based servers 304; servers 306; blade servers 308; storage devices 311; and networks and networking components 312. In some embodiments, software components include network application server software 314 and database software 316.

Virtualization layer 320 provides an abstraction layer from which the following examples of virtual entities may be provided: virtual servers 322; virtual storage 324; virtual networks 326, including virtual private networks; virtual applications and operating systems 328; and virtual clients 330.

In one example, management layer 340 may provide the functions described below. Resource provisioning 342 provides dynamic procurement of computing resources and other resources that are utilized to perform tasks within the cloud computing environment. Metering and Pricing 344 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 346 provides access to the cloud computing environment for consumers and system administrators. Service level management 348 provides cloud computing resource allocation and management such that required service levels are met. Service Level Agreement (SLA) planning and fulfillment 350 provide pre-arrangement for, and procurement of, cloud computing resources for which a future requirement is anticipated in accordance with an SLA.

Workloads layer 360 provides examples of functionality for which the cloud computing environment may be utilized. Examples of workloads and functions which may be provided from this layer include: mapping and navigation 362; software development and lifecycle management 364; virtual classroom education delivery 366; data analytics processing 368; transaction processing 370; and classification of genes based on DNA sequence signatures 372.

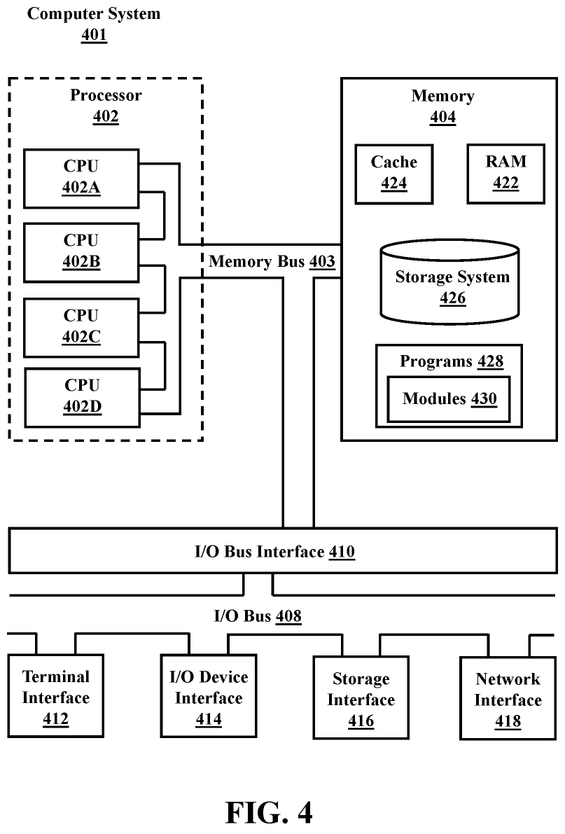

FIG. 4, illustrated is a high-level block diagram of an example computer system 401 that may be used in implementing one or more of the methods, tools, and modules, and any related functions, described herein (e.g., using one or more processor circuits or computer processors of the computer), in accordance with embodiments of the present disclosure. In some embodiments, the major components of the computer system 401 may comprise one or more CPUs 402, a memory subsystem 404, a terminal interface 412, a storage interface 416, an I/O (Input/Output) device interface 414, and a network interface 418, all of which may be communicatively coupled, directly or indirectly, for inter-component communication via a memory bus 403, an I/O bus 408, and an I/O bus interface unit 410.

The computer system 401 may contain one or more general-purpose programmable central processing units (CPUs) 402A, 402B, 402C, and 402D, herein generically referred to as the CPU 402. In some embodiments, the computer system 401 may contain multiple processors typical of a relatively large system; however, in other embodiments the computer system 401 may alternatively be a single CPU system. Each CPU 402 may execute instructions stored in the memory subsystem 404 and may include one or more levels of on-board cache.

System memory 404 may include computer system readable media in the form of volatile memory, such as random access memory (RAM) 422 or cache memory 424. Computer system 401 may further include other removable/non-removable, volatile/non-volatile computer system storage media. By way of example only, storage system 426 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, memory 404 can include flash memory, e.g., a flash memory stick drive or a flash drive. Memory devices can be connected to memory bus 403 by one or more data media interfaces. The memory 404 may include at least one program product having a set (e.g., at least one) of program modules that are configured to carry out the functions of various embodiments.

One or more programs/utilities 428, each having at least one set of program modules 430 may be stored in memory 404. The programs/utilities 428 may include a hypervisor (also referred to as a virtual machine monitor), one or more operating systems, one or more application programs, other program modules, and program data. Each of the operating systems, one or more application programs, other program modules, and program data or some combination thereof, may include an implementation of a networking environment. Programs 428 and/or program modules 430 generally perform the functions or methodologies of various embodiments.

Although the memory bus 403 is shown in FIG. 4 as a single bus structure providing a direct communication path among the CPUs 402, the memory subsystem 404, and the I/O bus interface 410, the memory bus 403 may, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 410 and the I/O bus 408 are shown as single respective units, the computer system 401 may, in some embodiments, contain multiple I/O bus interface units 410, multiple I/O buses 408, or both. Further, while multiple I/O interface units are shown, which separate the I/O bus 408 from various communications paths running to the various I/O devices, in other embodiments some or all of the I/O devices may be connected directly to one or more system I/O buses.

In some embodiments, the computer system 401 may be a multi-user mainframe computer system, a single-user system, or a server computer or similar device that has little or no direct user interface, but receives requests from other computer systems (clients). Further, in some embodiments, the computer system 401 may be implemented as a desktop computer, portable computer, laptop or notebook computer, tablet computer, pocket computer, telephone, smartphone, network switches or routers, or any other appropriate type of electronic device.

It is noted that FIG. 4 is intended to depict the representative major components of an exemplary computer system 401. In some embodiments, however, individual components may have greater or lesser complexity than as represented in FIG. 4, components other than or in addition to those shown in FIG. 4 may be present, and the number, type, and configuration of such components may vary.

As discussed in more detail herein, it is contemplated that some or all of the operations of some of the embodiments of methods described herein may be performed in alternative orders or may not be performed at all; furthermore, multiple operations may occur at the same time or as an internal part of a larger process.

The present disclosure may be a system, a method, and/or a computer program product at any possible technical detail level of integration. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Although the present disclosure has been described in terms of specific embodiments, it is anticipated that alterations and modification thereof will become apparent to the skilled in the art. Therefore, it is intended that the following claims be interpreted as covering all such alterations and modifications as fall within the true spirit and scope of the disclosure.

What is claimed is:

1. A computer-implemented method, the method comprising:
receiving, by a processor, DNA data associated with a DNA sequence; and
classifying the DNA sequence as exhibiting circadian behavior utilizing a graph deep learning algorithm, wherein the graph deep learning algorithm is trained utilizing a combination of features of a human interactome, features of DNA sequences associated with genes identified as exhibiting circadian behavior, and features of DNA sequences associated with genes identified as not exhibiting circadian behavior.

2. The method of claim 1, wherein the graph deep learning algorithm utilizes a graph convolutional network.

3. The method of claim 2, further comprising:
training the graph convolutional network utilizing k-mer spectra representing the DNA sequences associated with genes identified as exhibiting circadian behavior and k-mer spectra representing the DNA sequences associated with genes identified as not exhibiting circadian behavior.

4. The method of claim 3, wherein training the graphical convolution network further comprises:
utilizing a human interactome having elements of DNA sequences associated with genes identified as exhibiting circadian behavior and elements of DNA sequences associated with genes identified as not exhibiting circadian behavior mapped to the human interactome.

5. The method of claim 1, wherein training the graph deep learning algorithm further comprises:
identifying the genes exhibiting circadian behavior;
obtaining DNA sequences associated with the genes exhibiting circadian behavior;
translating the DNA sequences associated with the genes exhibiting circadian behavior to a representation suitable for machine learning;
identifying the genes not exhibiting circadian behavior;
obtaining DNA sequences associated with the genes not exhibiting circadian behavior; and
translating the DNA sequences associated with the genes not exhibiting circadian behavior to a representation suitable for machine learning.

6. The method of claim 5, wherein training the graph deep learning algorithm further comprises:
generating a mapping of the DNA sequences associated with the genes exhibiting circadian behavior and DNA sequences associated with the genes not exhibiting circadian behavior to the human interactome; and
translating the generated mapping to a representation suitable for machine learning.

7. The method of claim 1, wherein the DNA sequence is a single nucleotide polymorphism altered DNA sequence.

8. A system comprising:
a memory; and
a processor in communication with the memory, the processor being configured to perform operations comprising:
receiving DNA data associated with a DNA sequence; and
classifying the DNA sequence as exhibiting circadian behavior utilizing a graph deep learning algorithm, wherein the graph deep learning algorithm is trained utilizing a combination of features of a human interactome, features of DNA sequences associated with genes identified as exhibiting circadian behavior, and features of DNA sequences associated with genes identified as not exhibiting circadian behavior.

9. The system of claim 8, wherein the graph deep learning algorithm utilizes a graph convolutional network.

10. The system of claim 9, wherein the graph convolutional network is trained utilizing k-mer spectra representing the DNA sequences associated with genes identified as exhibiting circadian behavior and k-mer spectra representing the DNA sequences associated with genes identified as not exhibiting circadian behavior.

11. The system of claim 10, wherein the graph convolutional network is trained utilizing a human interactome having elements of DNA sequences associated with genes identified as exhibiting circadian behavior and elements of DNA sequences associated with genes identified as not exhibiting circadian behavior mapped to the human interactome.

12. The system of claim 8, wherein training the graph deep learning algorithm further comprises:
identifying the genes exhibiting circadian behavior;
obtaining DNA sequences associated with the genes exhibiting circadian behavior;
translating the DNA sequences associated with the genes exhibiting circadian behavior to a representation suitable for machine learning;
identifying the genes not exhibiting circadian behavior;
obtaining DNA sequences associated with the genes not exhibiting circadian behavior; and
translating the DNA sequences associated with the genes not exhibiting circadian behavior to a representation suitable for machine learning.

13. The system of claim 12, wherein training the graph deep learning algorithm further comprises:
generating a mapping of the DNA sequences associated with the genes exhibiting circadian behavior and DNA sequences associated with the genes not exhibiting circadian behavior to the human interactome; and
translating the generated mapping to a representation suitable for machine learning.

14. A computer program product comprising a computer readable storage medium having program instructions embodied therewith, the program instructions executable by a processor to cause the processor to perform operations, the operations comprising:
receiving DNA data associated with a DNA sequence; and
classifying the DNA sequence as exhibiting circadian behavior utilizing a graph deep learning algorithm, wherein the graph deep learning algorithm is trained utilizing a combination of features of a human interactome, features of DNA sequences associated with genes identified as exhibiting circadian behavior, and features of DNA sequences associated with genes identified as not exhibiting circadian behavior.

15. The computer program product of claim 14, wherein the graph deep learning algorithm utilizes a graph convolutional network.

16. The computer program product of claim 15, the processor being further configured to perform operations comprising: training the graph convolutional network utilizing k-mer spectra representing the DNA sequences associated with genes identified as exhibiting circadian behavior and k-mer spectra representing the DNA sequences associated with genes identified as not exhibiting circadian behavior.

17. The computer program product of claim 16, the processor being further configured to perform operations comprising: training the graphical convolution network utilizing a human interactome having elements of DNA sequences associated with genes identified as exhibiting circadian behavior and elements of DNA sequences associated with genes identified as not exhibiting circadian behavior mapped to the human interactome.

18. The computer program product of claim 14, wherein training the graph deep learning algorithm further comprises:
identifying the genes exhibiting circadian behavior;
obtaining DNA sequences associated with the genes exhibiting circadian behavior;
translating the DNA sequences associated with the genes exhibiting circadian behavior to a representation suitable for machine learning;
identifying the genes not exhibiting circadian behavior; and
obtaining DNA sequences associated with the genes not exhibiting circadian behavior;
translating the DNA sequences associated with the genes not exhibiting circadian behavior to a representation suitable for machine learning.

19. The computer program product of claim 18, wherein training the graph deep learning algorithm further comprises:
generating a mapping of the DNA sequences associated with the genes exhibiting circadian behavior and DNA sequences associated with the genes not exhibiting circadian behavior to the human interactome; and
translating the generated mapping to a representation suitable for machine learning.

20. The computer program product of claim 14, wherein the DNA sequence is a single nucleotide polymorphism altered DNA sequence.
